(19) United States
(12) Patent Application Publication
(10) Pub. No.: US 2026/0252761 A1
(43) Pub. Date: Aug. 27, 2026

(54) SEMICONDUCTOR PROCESS SIMULATION METHOD AND SYSTEM

(71) Applicant: Samsung Electronics Co., Ltd., Suwon-si (KR)

(72) Inventors: Hyoungsoo Ko, Suwon-si (KR); Taegeun Kim, Pohang-si (KR); Sejin Kim, Suwon-si (KR); Donghyun You, Pohang-si (KR); Sangseung Lee, Incheon-si (KR); Jaemyung Choe, Suwon-si (KR)

(73) Assignees: Inha University Research And Business Foundation, Incheon-si (KR); POSTECH RESEARCH AND BUSINESS DEVELOPMENT FOUNDATION, Gyeongsangbuk-do (KR)

(21) Appl. No.: 19/434,763

(22) Filed: Dec. 29, 2025

(30) Foreign Application Priority Data
Feb. 27, 2025 (KR) 10-2025-0026021

(51) Int. Cl. G06F 30/20; G16C 20/10

(52) U.S. Cl. CPC G06F 30/20; G16C 20/10

(57) ABSTRACT

A method includes generating a pattern factor database, dividing a wafer region into a plurality of divided regions, and generating a reactor scale simulation result by repeatedly performing a reactor scale simulation based on the pattern factor database, wherein the reactor scale simulation includes detecting a plurality of pattern factor values from the pattern factor database, updating a boundary condition of the reactor scale simulation based on the detected pattern factor values, and calculating the reaction counts for each of the plurality of divided regions.

### CROSS-REFERENCE TO RELATED APPLICATION

This application is based on and claims priority under 35 U.S.C. § 119 to Korean Patent Application No. 10-2025-0026021, filed on Feb. 27, 2025, in the Korean Intellectual Property Office, the disclosure of which is incorporated by reference herein in its entirety.

### BACKGROUND

Yields in semiconductor manufacturing processes may be affected by process variations occurring on surfaces of wafers inside reactors. Many semiconductor manufacturers have utilized computational fluid dynamics (CFD) simulations to predict yields. However, it is difficult to sufficiently reflect the effects of nano pattern shapes on processes, behavior of continuums, and changes in shape of patterns in real time, by using only this CFD-based simulation. Due to these difficulties, there may be limitations in predicting process variations by using process simulation.

One approach to addressing the issues described above is to use multi-scale bridging technology to exchange information between macro-scale facilities (e.g., reactors) and micro-scale nano patterns formed on wafers in real time and integrally interpret the information. However, implementation of the multi-scale bridging technology requires high computation costs.

### SUMMARY

The present disclosure relates to a semiconductor process simulation, and more particularly, to a multiscale-based semiconductor process simulation considering both development at a feature scale and development at a reactor scale.

Aspects of the present disclosure provide a method of reducing computation costs required for performing a semiconductor process simulation while improving accuracy of the semiconductor process simulation.

Aspects of the present disclosure are not limited to the technical objectives stated above, and other technical objectives not described herein are clearly understood by those skilled in the art from the following descriptions.

According to an aspect of the present disclosure, there is provided a semiconductor process simulation method including generating a pattern factor database based on reaction count-pattern factor information that represents an available reaction amount of a pattern according to a reaction count of a chemical reaction occurring on a surface of a target pattern, dividing a wafer region into a plurality of divided regions, and generating a reactor scale simulation result by repeatedly performing a reactor scale simulation based on the pattern factor database, wherein the reactor scale simulation includes detecting, from the pattern factor database, a plurality of pattern factor values corresponding to reaction counts of each of the plurality of divided regions, updating a boundary condition of the reactor scale simulation based on the detected pattern factor values, and performing the reactor scale simulation based on the updated boundary condition and calculating the reaction counts for each of the plurality of divided regions.

According to another aspect of the present disclosure, there is provided a semiconductor process simulation system including memory configured to store a program for performing a semiconductor process simulation, and a processor configured to execute the program stored in the memory, wherein the processor is configured to generate a pattern factor database representing an available reaction amount of a pattern according to a reaction count of a chemical reaction occurring on a surface of a target pattern, divide a wafer region into a plurality of divided regions, and generate a reactor scale simulation result by repeatedly performing a reactor scale simulation based on the pattern factor database, and wherein, when performing the reactor scale simulation, the processor is configured to detect, from the pattern factor database, a plurality of pattern factor values corresponding to reaction counts of each of the plurality of divided regions, update a boundary condition of the reactor scale simulation based on the detected pattern factor values, and perform the reactor scale simulation based on the updated boundary condition and calculate the reaction counts for each of the plurality of divided regions.

According to another aspect of the present disclosure, there is provided a computer-readable non-transitory storage medium configured to store commands, when executed by a processor, to make the processor perform a semiconductor process simulation, wherein the semiconductor process simulation includes generating a pattern factor database representing an available reaction amount of a pattern according to a reaction count of a chemical reaction occurring on a surface of a target pattern, dividing a wafer region into a plurality of divided regions, and generating a simulation result by repeatedly performing a simulation based on the pattern factor database, wherein the generating of the simulation result includes detecting, from the pattern factor database, a plurality of pattern factor values corresponding to reaction counts of each of the plurality of divided regions, updating a boundary condition of the reactor scale simulation based on the detected pattern factor values, and performing the reactor scale simulation based on the updated boundary condition and calculating the reaction counts for each of the plurality of divided regions.

### BRIEF DESCRIPTION OF THE DRAWINGS

Implementations will be more clearly understood from the following detailed description taken in conjunction with the accompanying drawings in which:

FIG. 1 is a diagram showing a semiconductor process simulation system according to an implementation;

FIG. 2 is a diagram showing a simulation device according to an implementation;

FIG. 3 is a flowchart illustrating a semiconductor process simulation method according to implementations;

FIGS. 4A to 4D are diagrams illustrating simulation parameters according to implementations;

FIG. 5 is a flowchart illustrating a semiconductor process simulation method according to implementations;

FIG. 6 is a flowchart illustrating a semiconductor process simulation method according to implementations;

FIGS. 7A to 7E are diagrams illustrating a process of generating a pattern factor database;

FIGS. 8A and 8B are diagrams illustrating reaction count-pattern factor information according to implementations;

FIG. 9 is a flowchart illustrating a semiconductor process simulation method according to implementations;

FIGS. 10A and 10B are diagrams illustrating a reactor scale simulation performed based on a pattern factor database; and

FIG. 11 is a block diagram illustrating a computer system according to an implementation.

### DETAILED DESCRIPTION

Hereinafter, some implementations are described in detail with reference to the accompanying drawings. In descriptions with reference to drawings, the same reference numerals are given to the same or corresponding components, and repeated descriptions thereof are omitted.

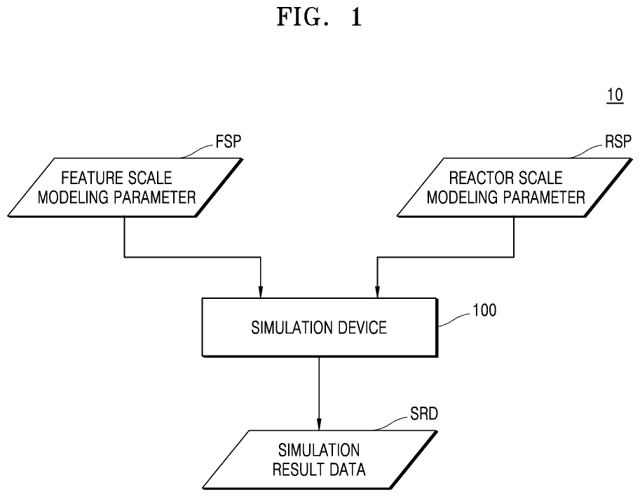

FIG. 1 is a diagram showing a semiconductor process simulation system 10 according to an implementation.

Referring to FIG. 1, the semiconductor process simulation system 10 may include a system for calculating process variations in a semiconductor process. To calculate the process variations in a semiconductor process, the semiconductor process simulation system 10 may include a simulation device 100. The simulation device 100 may perform simulation based on a feature scale modeling parameter FSP and a reactor scale modeling parameter RSP, and may generate simulation result data SRD.

In the present disclosure, the semiconductor process to be simulated is assumed to be a semiconductor deposition process, but this is only for illustrative purposes and is not intended to limit the present disclosure.

As used herein, unless otherwise specified (e.g., an “actual wafer” or a “real wafer”), a wafer may represent a wafer to be simulated inside the simulation device 100. The wafer to be simulated may represent a data structure that imitates a real wafer subjected to an actual semiconductor process. In some implementations, the wafer to be simulated may be referred to as a target wafer.

In an implementation, the simulation device 100 may simulate a deposition process, which is a process of forming a film on a wafer surface, and may simulate, for example, a tungsten chemical vapor deposition (CVD) process as shown in [Reaction equation 1] below. The example of tungsten CVD is provided for illustrative purposes only and is not intended to limit the present disclosure.

$${WF}_{6} ( g ) + 3 {H}_{2} ( g ) \to W ( s ) + 6 HF ( g ) \qquad [ Reaction equation 1 ]$$

As used herein, a process variation may represent the degree of uniformity of physical and electrical characteristics (e.g., the thickness of a layer formed on a wafer, the shape of a pattern, etc.) of the wafer after a process simulation is performed. The simulation result data SRD may include information about the process variation obtained by the simulation device 100 performing the simulation.

In the present disclosure, the feature scale may represent a scale used to analyze semiconductor processes in terms of microscopic aspects. For example, in feature scale analysis, it is possible to simulate localized reactions, surface reactions, and shape changes that occur in structures of nm to μm scale, such as trenches, vias, and holes in fine patterns.

In the present disclosure, the simulation based on the feature scale may be referred to as the feature scale simulation.

In the present disclosure, the reactor scale may represent a scale used to totally analyze the reactor (in some implementations, the reactor may be referred to as a chamber) in which the semiconductor process is performed, in terms of macroscopic aspects. For example, in reactor scale analysis, it is possible to simulate phenomena occurring inside the reactor, such as fluid flow, temperature distribution, pressure, gas concentration, and by-product discharge paths inside the reactor, while considering the structure of the reactor and the locations and shapes of wafers arranged inside the reactor.

In the present disclosure, the simulation based on the reactor scale may be referred to as the reactor scale simulation.

In an implementation, the simulation device 100 may divide the target wafer, which is to be analyzed in the reactor scale simulation, into a plurality of divided regions. The simulation device 100 may perform a simulation corresponding to the [Reaction equation 1] on each of the divided regions, thereby calculating a reaction amount (i.e., a reaction count) occurring in each of the divided regions.

The feature scale modeling parameter FSP may include parameters used to analyze, in the feature scale, chemical reactions (e.g., [Reaction equation 1]) that occur in semiconductor processes with respect to fine patterns formed on actual wafers.

In the present disclosure, a target pattern may represent a data structure that simulates the fine pattern formed on the actual wafer. For example, the target pattern may represent a trench pattern formed in the wafer.

In an implementation, the simulation device 100 may perform the feature scale simulation for the target pattern by using the feature scale modeling parameter FSP. A process considered when the feature scale simulation is performed may simulate a process performed on the actual wafer.

The reactor scale modeling parameter RSP may include parameters used to analyze, in the reactor scale, chemical reactions (e.g., [Reaction equation 1]) that occur in the semiconductor process, inside the reactor in which the actual semiconductor process is performed.

In an implementation, the simulation device 100 may perform the reactor scale simulation by using the reactor scale modeling parameter RSP. A process considered when the reactor scale simulation is performed may simulate a process performed on the actual wafer.

To improve the accuracy of reactor scale analysis, the effects of feature scale may be considered. However, due to the difference in units between the analysis based on feature scale and the analysis based on reactor scale, it may need significantly large amounts of computing resources to perform simulations in which the effects of feature scale are directly reflected in the reactor scale.

The simulation device 100 according to an implementation may perform the feature scale-based simulation in advance when performing a semiconductor process simulation, thereby generating a database that includes pattern factor values for reflecting the effects of nano patterns, existing on the wafer, in the reactor scale simulation. The simulation device 100 may perform the reactor scale simulation while considering the effects of the feature scale, on the basis of the database generated by the feature scale-based simulation. Accordingly, according to an implementation, the accuracy of semiconductor process simulation may be improved, and the computation costs for the semiconductor process simulation may be reduced.

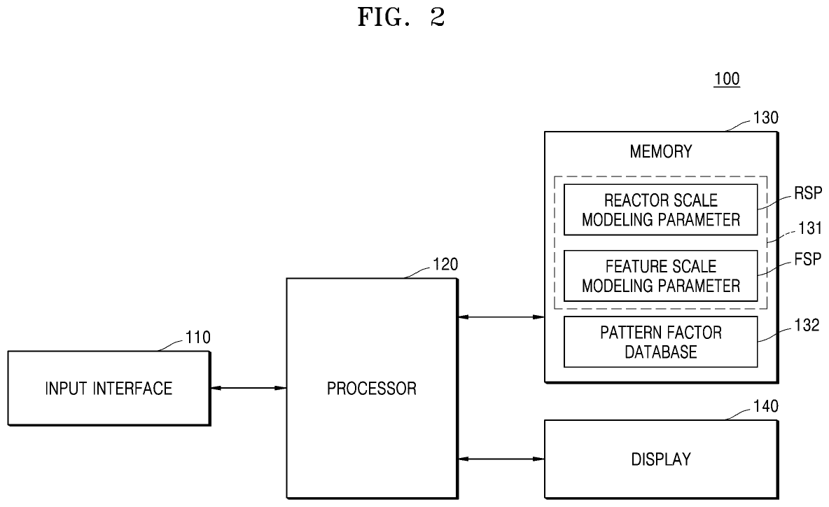

FIG. 2 is a diagram showing the simulation device 100 according to an implementation. FIG. 2 may be described with reference to FIG. 1, and repeated descriptions thereof may be omitted.

Referring to FIG. 2, the simulation device 100 may include an input interface 110, a processor 120, memory 130, and a display 140.

The input interface 110 may function as a path for various other external devices connected to the simulation device 100. In some implementations, the input interface 110 may receive input information that includes input values from a user, etc.

The input interface 110 may communicate with the processor 120. In some implementations, the input interface 110 may provide the processor 120 with the input information received from an external source.

The input interface 110 may include at least one of a wired/wireless headset port, an external charger port, a wired/wireless data port, a memory card port, a port for connection to a device provided with a subscriber identification module (SIM), an audio input/output (I/O) port, a video I/O port, and an earphones port.

The processor 120 may process various functions performed by the simulation device 100 or control these functions. The processor 120 may control an operation of the input interface 110, an operation of the memory 130, and/or an operation of the display 140.

The processor 120 may control at least some of components shown in FIG. 2 to run an application program stored in the memory 130. Furthermore, the processor 120 may combine and operate at least two of the components in the simulation device 100 to run the application program.

The processor 120 may generally control all operations of the simulation device 100 in addition to operations relating to the application program. The processor 120 may provide or process appropriate information or functions to a user, by processing signals, data, information, etc. input or output via the components described above or by executing application programs stored in the memory 130.

The processor 120 may load a simulation parameter 131 and a pattern factor database 132 from the memory 130.

The processor 120 may simulate a semiconductor process on the basis of the simulation parameter 131 and may output, as the simulation result data SRD, the process variation formed on the wafer by the simulation.

The processor 120 may use the simulation parameter 131 to calculate the reaction count that represents the reaction amount of the chemical reaction expressed as shown in [Equation 1] below. [Equation 1] may be defined as a pressure value of a first material (e.g., tungsten hexafluoride), a pressure value of a second material (e.g., hydrogen), activation energy of [Reaction equation 1], and/or a pattern factor value.

In some implementations, the processor 120 may include one or more processors. In some implementations, all of the functions of the processor 120 described herein may be performed by a single processor. In other implementations, the functions of the processor 120 may be distributed among multiple processors (e.g., one processor performs a subset of the functions of the processor 120 while one or more other processors perform the remaining functions of the processor 120.)

In some implementations, an equation for calculating the reaction count may be expressed as shown in [Equation 1] below. Here, [Equation 1] may be a mathematical equation corresponding to [Reaction equation 1]. In some implementations, [Equation 1] may be referred to as the Arrhenius equation.

$$R = α {k}_{0} exp ( - \frac{{E}_{a}}{RT} ) \frac{{P}_{{H}_{2}}^{0.5} {P}_{{WF}_{6}}}{1 + 1000 {P}_{{WF}_{6}}} \qquad [ Equation 1 ]$$

In [Equation 1], α may represent the pattern factor value. When α=1, it may indicate that the pattern is flat. Also, when α increases, it may indicate that the pattern is not flat. For example, as a increases, the depth of a trench of a pattern may increase. In some implementations, the pattern factor value may be referred to as a reaction coefficient. Also, in some implementations, the pattern factor value may be referred to as a 3D pattern factor value.

In [Equation 1], k₀ may represent a constant for [Reaction equation 1] defined when α=1.

In [Equation 1], Eₐ may represent the activation energy in [Reaction equation 1].

In [Equation 1], R may represent the gas constant.

In [Equation 1], T may represent the temperature of the chemical reaction equation by [Reaction equation 1].

In [Equation 1], P_{H}_{₂} may represent the pressure of hydrogen gas.

In [Equation 1], P_{WF}_{₆} may represent the pressure of tungsten hexafluoride gas.

In some implementations, the temperature, the pressure of hydrogen gas, and the pressure of tungsten hexafluoride gas may be preset values or values input from an external source by a user, and may be included in the simulation parameter 131.

The memory 130 may store data for supporting various functions of the simulation device 100. The memory 130 may store a plurality of application programs or applications executed by the simulation device 100, data for the operation of the simulation device 100, and commands. The memory 130 may be implemented as a memory device.

The memory 130 may store the simulation parameter 131 and the pattern factor database 132. The simulation parameter 131 may include the reactor scale modeling parameter RSP and the feature scale modeling parameter FSP.

In an implementation, the pattern factor database 132 may be generated by performing a simulation based on the feature scale modeling parameter FSP. In some implementations, the pattern factor database 132 may be referred to as a lookup table, a database, or a pattern factor table.

The simulation device 100 may further include a communication module for performing wired/wireless communication with external devices.

The wireless communication may include, for example, wireless LAN (WLAN), wireless fidelity (Wi-Fi), Wi-Fi direct, digital living network alliance (DLNA), wireless broadband (WiBro), world interoperability for microwave access (WiMAX), high-speed downlink packet access (HSDPA), high-speed uplink packet access (HSUPA), long-term evolution (LTE), and LTE-Advanced (LTE-A).

The short range communication may include, for example, Bluetooth™, radio frequency identification (RFID), infrared data association (IrDA), ultra wideband (UWB), ZigBee, near field communication (NFC), Wi-Fi, Wi-Fi direct, and wireless universal serial bus (Wireless USB) technology.

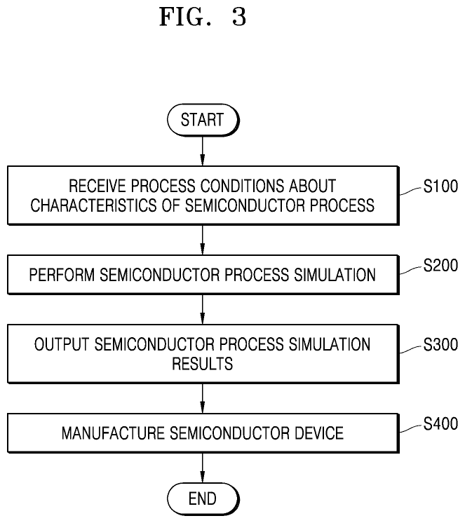

FIG. 3 is a flowchart illustrating a semiconductor process simulation method according to some implementations. FIG. 3 may be described with reference to FIGS. 1 and 2, and repeated descriptions thereof may be omitted.

Referring to FIG. 3, in operation S100, the simulation device 100 may receive a process condition relating to the characteristics of a semiconductor process from an external source. Process conditions received by the simulation device 100 may be reflected in feature scale modeling parameters FSP and the reactor scale modeling parameters RSP and may simulate actual process conditions.

In an implementation, the process conditions reflected in the feature scale modeling parameters FSP are used to simulate semiconductor processes performed on fine patterns and may include microscopic process conditions, such as a temperature, a gas concentration, a diffusion rate of gas, and a surface reaction on the fine patterns.

In an implementation, the process conditions reflected in the reactor scale modeling parameters RSP are used to entirely simulate the reactor and may include macroscopic process conditions, such as a flow pattern, temperature distribution, pressure, a gas flow rate, and global concentration inside the reactor.

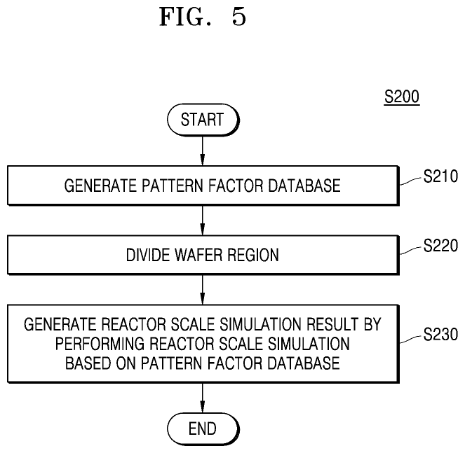

In operation S200, the simulation device 100 may perform simulation by reflecting the received process conditions in the feature scale modeling parameters FSP and the reactor scale modeling parameters RSP. Operation S200 is described in detail with reference to FIG. 5.

In operation S300, the simulation device 100 may output simulation results on the display 140.

In operation S400, an actual semiconductor device may be manufactured. The semiconductor device may be manufactured based on the simulation results obtained by operation S300.

In an implementation, based on the simulation results output in operation S300, a user may modify process conditions or layout designs used in the actual manufacturing process of the semiconductor device, thereby improving the manufacturing yield of semiconductor devices.

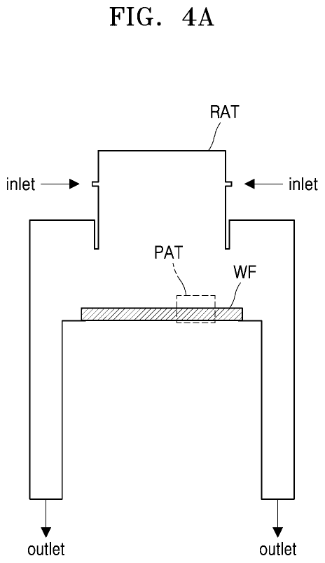

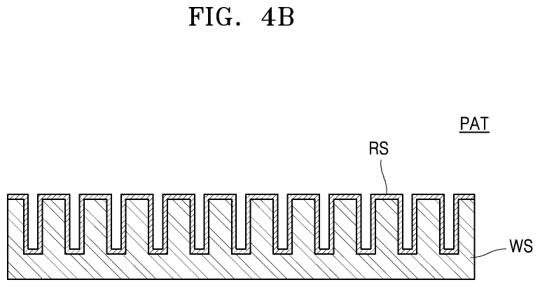

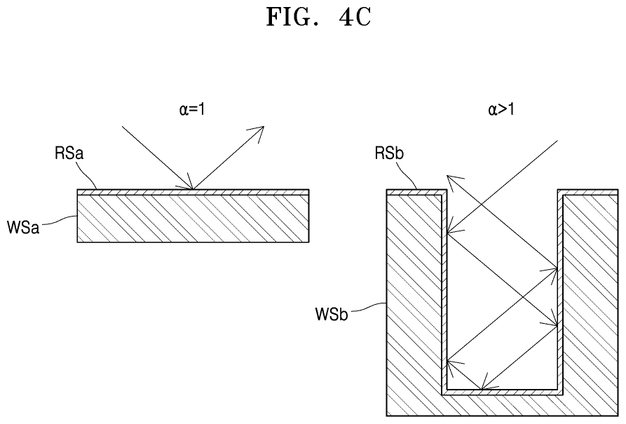

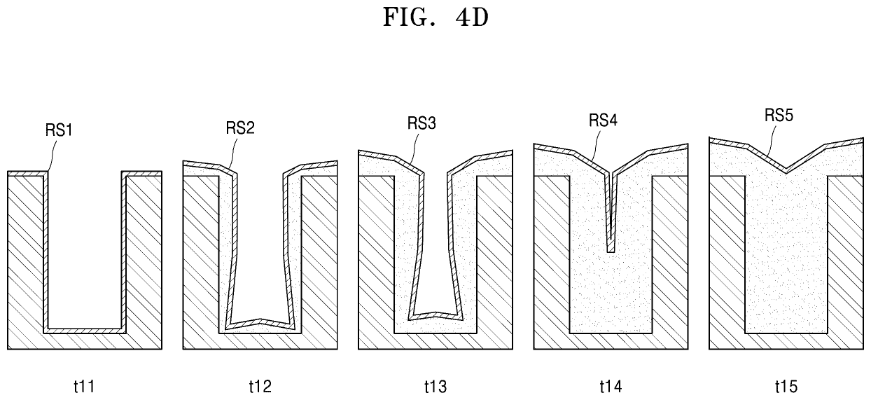

FIGS. 4A to 4D are diagrams illustrating simulation parameters according to some implementations. FIGS. 4A to 4D may be described with reference to FIGS. 1 and 2, and repeated descriptions thereof may be omitted.

FIG. 4A shows a reactor RAT that is to be simulated by using the reactor scale modeling parameters RSP. The reactor scale modeling parameter RSP may represent parameters for simulating chemical reactions occurring in the reactor RAT at a macroscopic scale.

Referring to FIG. 4A, the reactor RAT may have an inlet through which a reaction material (e.g., tungsten hexafluoride and hydrogen) is supplied into the reactor RAT. In addition, the reactor RAT may have an outlet through which by-products generated after the chemical reaction or unused residual gases are discharged from the reactor RAT. A wafer WF may be placed inside the reactor RAT, and the chemical reaction shown in [Reaction equation 1] may occur due to the materials supplied through the inlet of the reactor RAT.

In some implementations, a region occupied by the wafer WF in the reactor scale modeling parameters RSP may be referred to as a wafer region.

Regarding the reactor scale modeling parameters RSP, a huge amount of computing resources may be required to specifically simulate a microscopic structure (e.g., a trench formed in a real wafer) of a pattern PAT shown in FIG. 4B on the wafer WF at a macroscopic scale. In the reactor scale modeling parameters RSP according to an implementation, reactor scale simulations may be performed based on boundary conditions reflecting the effect of the pattern PAT, rather than simulating the microscopic structure of the pattern PAT. Here, the boundary conditions may represent pattern factor values corresponding to the thickness of the film formed on the pattern PAT.

FIG. 4B shows the pattern PAT that is to be simulated by using the feature scale modeling parameters FSP. The feature scale modeling parameters FSP may represent parameters for simulating chemical reactions occurring in the pattern PAT at a microscopic scale.

Referring to FIG. 4B, the pattern PAT may be shown from a microscopic perspective as part of the wafer WF of FIG. 4A. The pattern PAT may include a plurality of trenches formed in a wafer surface WS. Due to the trenches formed in the wafer surface WS, the area of a reaction surface RS, on which the reaction according to [Reaction equation 1] occurs, may increase.

In the simulation using the feature scale modeling parameters FSP, the change in the thickness of a tungsten film formed on the reaction surface RS may be simulated over a process time, as shown in FIG. 4D.

FIG. 4C is a diagram illustrating the pattern factor value. Referring to FIG. 4C, when the pattern factor value is 1, this may indicate that a wafer surface WSa is flat. When the pattern factor value is greater than 1, this may indicate that a wafer surface WSb is not flat. For example, as shown on the right figure of FIG. 4C, a trench may be formed in the wafer surface WSb.

When the wafer surface WSb is not flat, this may indicate that the area of contact between the material and the wafer surface WSb increases when a chemical reaction such as [Reaction equation 1] occurs. As shown in FIG. 4C, the number of collisions of the material with a reaction surface RSb when the pattern factor value is greater than 1 may be greater than the number of collisions of the material with a reaction surface RSa when the pattern factor value is 1.

In some implementations, a pattern having a pattern factor value of 1 may be referred to as a flat pattern.

Referring to FIG. 4D, the simulation device 100 may perform simulation, for example, for a preset simulation period of time (e.g., 20,000 sec) by using the feature scale modeling parameters FSP, and as a result, may calculate the cumulative reaction count over the elapsed process time. The cumulative reaction count may be calculated based on the thickness of the film formed between the wafer surface WS and the reaction surface. In an implementation, the thickness of the film may be proportional to the cumulative reaction count.

FIG. 4D shows five points in time during the simulation period of time, i.e., a first point in time t11 to a fifth point in time t15, and it is assumed that the points in time are separated from each other at intervals corresponding to a reference period of time (5,000 sec). Here, the first point in time t11 may represent the initial point in time when the simulation starts, and the fifth point in time t15 may represent the end point in time when the simulation is completed. However, this is an example for convenience of description and is not intended to limit the present disclosure.

The first point in time t11 represents a first reaction surface RS1, which is in an initial state of the reaction surface before a process is performed on the wafer surface WS.

The second point in time t12 represents a second reaction surface RS2, which is the reaction surface after the reference period of time has elapsed from the first point in time t11. The thickness of the film formed at the second point in time t12 may increase compared to the first point in time t11. In this case, as the thickness of the film increases, the state of the reaction surface may change from the first reaction surface RS1 to the second reaction surface RS2. Also, the area of the second reaction surface RS2 may be less than the area of the first reaction surface RS1.

The third point in time t13 represents a third reaction surface RS3, which is the reaction surface after the reference period of time has elapsed from the second point in time t12. The thickness of the film formed at the third point in time t13 may increase compared to the second point in time t12. In this case, as the thickness of the film increases, the state of the reaction surface may change from the second reaction surface RS2 to the third reaction surface RS3. Also, the area of the third reaction surface RS3 may be less than the area of the second reaction surface RS2.

The fourth point in time t14 represents a fourth reaction surface RS4, which is the reaction surface after the reference period of time has elapsed from the third point in time t13. The thickness of the film formed at the fourth point in time t14 may increase compared to the third point in time t13. In this case, as the thickness of the film increases, the state of the reaction surface may change from the third reaction surface RS3 to the fourth reaction surface RS4. Also, the area of the fourth reaction surface RS4 may be less than the area of the third reaction surface RS3.

The fifth point in time t15 represents a fifth reaction surface RS5, which is the reaction surface after the reference period of time has elapsed from the fourth point in time t14. The thickness of the film formed at the fifth point in time t15 may increase compared to the fourth point in time t14. In this case, as the thickness of the film increases, the state of the reaction surface may change from the fourth reaction surface RS4 to the fifth reaction surface RS5. Also, the area of the fifth reaction surface RS5 may be less than the area of the fourth reaction surface RS4.

The thickness of the film formed on the wafer surface WS may be proportional to the cumulative reaction count occurring on the wafer surface WS. The thickness of the film formed on the wafer surface WS may be inversely proportional to the area of the reaction surface.

In an implementation, the pattern factor value corresponding to the fifth reaction surface RS5 at the fifth point in time t15 may be a value that converges to the pattern factor value corresponding to the reaction surface RSa in FIG. 4C, and may be considered to be the same as the pattern factor value corresponding to the reaction surface RSa in FIG. 4C.

FIG. 5 is a flowchart illustrating a semiconductor process simulation method according to some implementations. FIG. 5 may be described with reference to FIGS. 1 and 2, and repeated descriptions thereof may be omitted.

Referring to FIG. 5, in operation S210, the simulation device 100 may use the feature scale modeling parameters FSP to perform the feature scale simulation, thereby generating the pattern factor database 132.

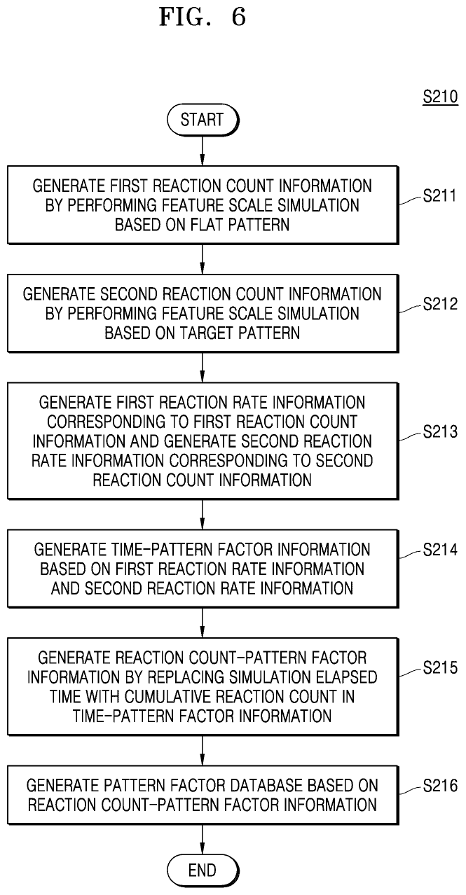

The pattern factor database 132 may be a database for representing the degree of flatness of a pattern according to the reaction count of the chemical reaction (e.g., the reaction according to [Reaction equation 1]) occurring on the surface of the target pattern, an available reaction amount of the pattern, or a possible reaction probability of the pattern. Operation S210 is described in detail with reference to FIG. 6.

In operation S220, the simulation device 100 may divide a wafer region included in the reactor scale modeling parameters RSP. Here, the region to be divided may be referred to as a divided region, and the wafer region may include a plurality of divided regions.

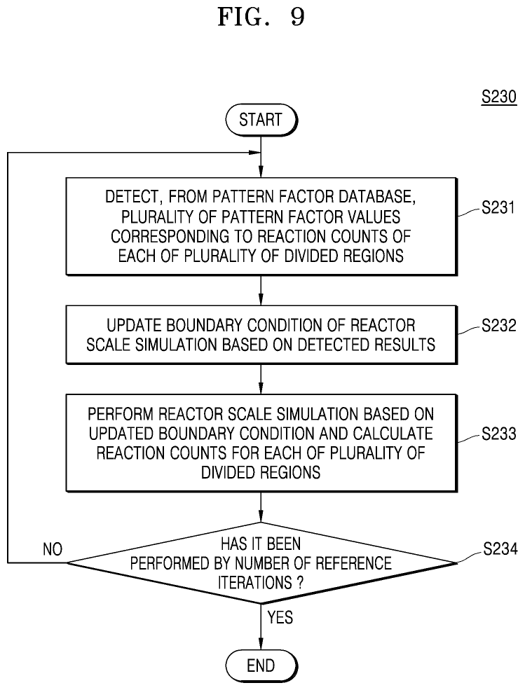

In operation S230, the simulation device 100 may generate a reactor scale simulation result by performing the reactor scale simulation based on the pattern factor database 132 and the reactor scale modeling parameters RSP, and the reactor scale simulation result may correspond to the simulation result data SRD. Operation S230 is described in detail with reference to FIG. 9.

In an implementation, the reactor scale simulation according to operation S230 may be performed repeatedly according to a preset reference value of iterations.

In an implementation, the reference value of iterations may represent a value stored in the memory 130 of the simulation device 100 or a value input from an external source.

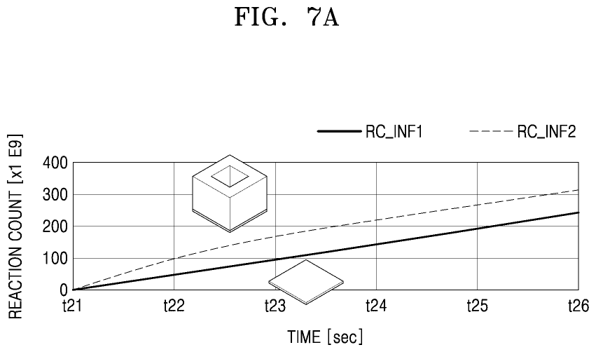

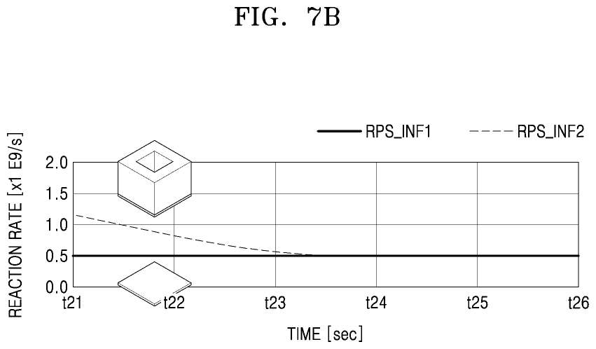

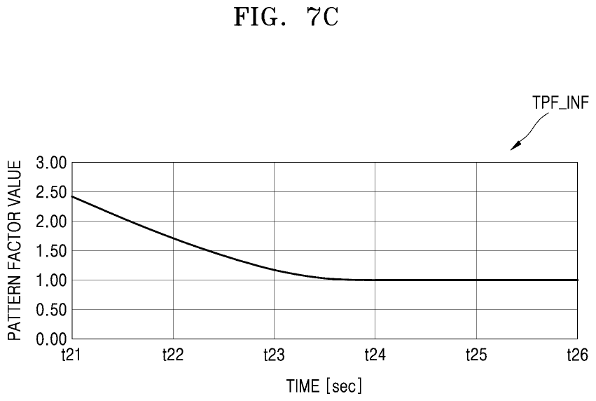

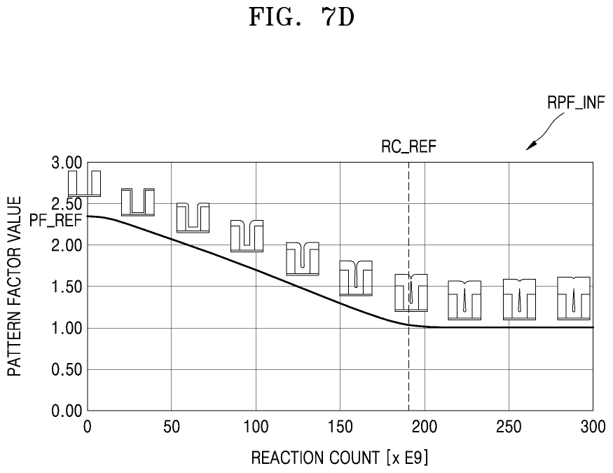

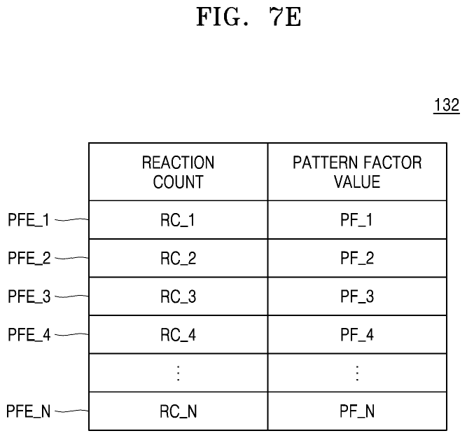

FIG. 6 is a flowchart illustrating a semiconductor process simulation method according to some implementations. Specifically, FIG. 6 may be a flowchart illustrating operation S210 of FIG. 5. FIGS. 7A to 7E are diagrams illustrating a process of generating the pattern factor database 132. FIGS. 6 and 7A to 7E may be described with reference to FIGS. 1 to 3, 4A to 4D, and 5, and repeated descriptions thereof may be omitted.

The values relating to time, reaction count, reaction rate, and pattern factor value shown on a first axis (x-axis) or a second axis (y-axis) of graphs in FIGS. 7A to 7D are only examples for illustrative purposes and are not intended to limit the present disclosure.

Referring to FIGS. 6 and 7A, in operation S211, the simulation device 100 may generate first reaction count information RC_INF1 by performing the feature scale simulation based on a flat pattern.

In an implementation, the structure of the flat pattern may correspond to the structure of the left pattern among the patterns shown in FIG. 4C.

In some implementations, the simulation device 100 may generate the first reaction count information RC_INF1 by simulating the chemical reaction according to [Reaction equation 1] at the feature scale on the basis of the flat pattern. The first reaction count information RC_INF1 may represent cumulative reaction counts that occur on the surface of the flat pattern as a function of simulation elapsed time, as shown in FIG. 7A.

In operation S212, the simulation device 100 may generate second reaction count information RC_INF2 by performing the feature scale simulation based on the target pattern.

In an implementation, the structure of the target pattern may correspond to the structure of the right pattern among the patterns shown in FIG. 4C.

In some implementations, the simulation device 100 may generate the second reaction count information RC_INF2 by simulating the chemical reaction according to [Reaction equation 1] at the feature scale on the basis of the target pattern. The second reaction count information RC_INF2 may represent cumulative reaction counts that occur on the surface of the target pattern as a function of simulation elapsed time, as shown in FIG. 7A.

Referring to FIGS. 6 and 7B, in operation S213, the simulation device 100 may generate first reaction rate information RPS_INF1 corresponding to the first reaction count information RC_INF1. The simulation device 100 may generate second reaction rate information RPS_INF2 corresponding to the second reaction count information RC_INF2.

In an implementation, the simulation device 100 may generate the first reaction rate information RPS_INF1 by differentiating the first reaction count information RC_INF1 according to time.

In an implementation, the simulation device 100 may generate the second reaction rate information RPS_INF2 by differentiating the second reaction count information RC_INF2 according to time.

Referring to FIGS. 6 and 7C, in operation S214, the simulation device 100 may generate time-pattern factor information TPF_INF, based on the first reaction rate information RPS_INF1 and the second reaction rate information RPS_INF2.

In an implementation, the simulation device 100 may obtain a pattern factor value by dividing a reaction rate value extracted from the second reaction rate information RPS_INF2 at a specific point in time by a reaction rate value extracted from the first reaction rate information RPS_INF1 at the same point in time.

In an implementation, the simulation device 100 may sample reaction rate values from the first reaction rate information RPS_INF1 and the second reaction rate information RPS_INF2 at every certain interval (e.g., 10 sec) during a simulation period of time (a period of time corresponding to a first point in time t21 to a sixth point in time t26). Also, the simulation device 100 may obtain the pattern factor values by performing the division operation described above and generate the time-pattern factor information TPF_INF based on the obtained pattern factor values.

Referring to FIGS. 6 and 7D, in operation S215, the simulation device 100 may generate reaction count-pattern factor information RPF_INF by replacing the simulation elapsed time (i.e., the x-axis of the graph in FIG. 7C) with the reaction count, based on the time-pattern factor information TPF_INF.

In an implementation, based on the reaction count-pattern factor information RPF_INF, when the reaction count is less than a reference reaction count RC_REF, the reaction count may be inversely proportional to the pattern factor value.

In an implementation, when the reaction count is greater than the reference reaction count RC_REF, the pattern factor value may remain constant.

In an implementation, when the reaction count in the reaction count-pattern factor information RPF_INF is 0, the pattern factor value may have a reference pattern factor value PF_REF. The reference pattern factor value PF_REF may be greater than 1 and stored in the simulation device 100 as a preset value.

In an implementation, the reference pattern factor value PF_REF and the reference reaction count RC_REF may vary depending on geometrical characteristics of the target pattern. For example, the values may vary depending on the depth of a pattern and the width of an opening at the top of the pattern (which may be referred to as a top opening in some implementations).

Referring to FIG. 6, in operation S216, the simulation device 100 may generate the pattern factor database 132 based on the reaction count-pattern factor information RPF_INF. The pattern factor database 132 is described in detail with reference to FIG. 7E.

Referring to FIG. 7E, the pattern factor database 132 may have a data structure corresponding to the reaction count-pattern factor information RPF_INF shown in FIG. 7D.

The pattern factor database 132 may include a plurality of pattern factor entries PFE_1 to PFE_N (where N is a natural number of 2 or more). Each of the plurality of pattern factor entries PFE_1 to PFE_N may include a reaction count value and a pattern factor value. The reaction count value may represent the number of cumulative reaction particles of the chemical reaction generated on the surface of the target pattern. The pattern factor value may represent the degree of flatness of the pattern.

In an implementation, first to Nth reaction count values RC_1 to RC_N may be arranged in ascending order. That is, the first reaction count value RC_1 may have the smallest value among the first to Nth reaction count values RC_1 to RC_N, and the Nth reaction count value RC_N may have the largest value among the first to Nth reaction count values RC_1 to RC_N. The first reaction count value RC_1 may be, for example, 0.

In an implementation, first to Nth pattern factor values PF_1 to PF_N may be arranged in descending order. That is, the first pattern factor value PF_1 may have the largest value among the first to Nth pattern factor values PF_1 to PF_N, and the Nth pattern factor value PF_N may have the smallest value among the first to Nth pattern factor values PF_1 to PF_N. The first pattern factor value PF_1 may represent, for example, the reference pattern factor value PF_REF.

In an implementation, the first pattern factor entry PFE_1 may include the first reaction count value RC_1 and the first pattern factor value PF_1. The first reaction count value RC_1 may include a value representing the cumulative reaction count until the first point in time during the feature scale simulation. The first pattern factor value PF_1 may include a pattern factor value corresponding to the first reaction count value RC_1 and may include a value inversely proportional to the thickness of the film formed at the first point in time.

In an implementation, the second pattern factor entry PFE_2 may include the second reaction count value RC_2 and the second pattern factor value PF_2. The second reaction count value RC_2 may include a value representing the cumulative reaction count until the second point in time during the feature scale simulation. The second point in time may be defined as the point in time after the first point in time. The second pattern factor value PF_2 may include a pattern factor value corresponding to the second reaction count value RC_2 and may include a value inversely proportional to the thickness of the film formed at the second point in time.

The descriptions of the first pattern factor entry PFE_1 may equally apply to the third pattern factor entry PFE_3 to the Nth pattern factor entry PFE_N, and thus, repeated descriptions thereof are omitted.

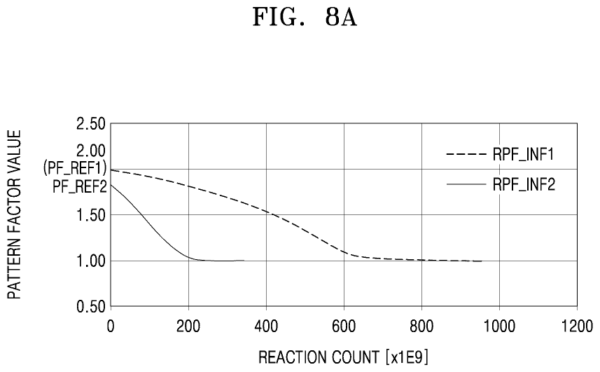

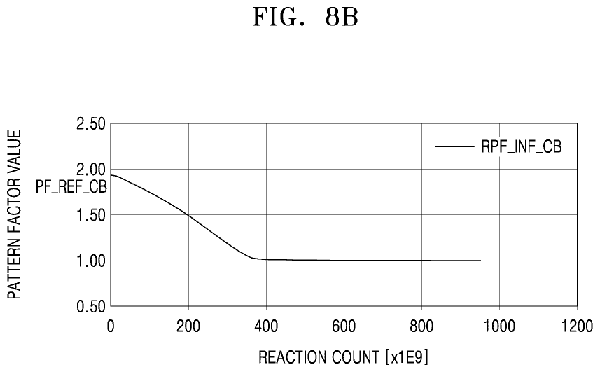

FIGS. 8A and 8B are diagrams illustrating the reaction count-pattern factor information according to some implementations. FIGS. 8A and 8B may be described with reference to FIGS. 1 to 3, 4A to 4D, 5, 6, and 7A to 7E, and repeated descriptions thereof may be omitted.

A plurality of fine patterns may exist on a real wafer. The plurality of fine patterns existing on the real wafer may have different shapes. Therefore, in order to derive accurate simulation results, aspects of the present disclosure may reflect the effects of fine patterns with different shapes. The simulation device 100 may simulate the plurality of fine patterns on the real wafer for each pattern, thereby generating reaction count-pattern factor information relating to each fine pattern. In other words, in the feature scale simulation, the target pattern to be simulated may be two or more. In FIG. 8A, it is assumed that the simulation device 100 performs the feature scale simulation on each of a first target pattern and a second target pattern. The example in which the simulation device 100 performs the feature scale simulation on two types of target patterns is given for illustrative purposes and is not intended to limit the present disclosure. The simulation device 100 may also perform the feature scale simulation on two or more types of target patterns existing on the real wafer.

Referring to FIG. 8A, the first target pattern may be different from the second target pattern. For example, the width of an opening at the top of the first target pattern may be greater than the width of an opening at the top of the second target pattern. Also, for example, the depth of the trench of the first target pattern may be greater than the depth of the trench of the second target pattern.

The simulation device 100 may perform the feature scale simulation on the first target pattern, as shown in FIGS. 7A to 7D, to generate first reaction count-pattern factor information RPF_INF1 corresponding to the first target pattern. Also, the simulation device 100 may perform the feature scale simulation on the second target pattern, as shown in FIGS. 7A to 7D, to generate second reaction count-pattern factor information RPF_INF2 corresponding to the second target pattern.

In an implementation, a reference reaction count corresponding to the first reaction count-pattern factor information RPF_INF1 may include a first reference pattern factor value PF_REF1. A reference reaction count corresponding to the second reaction count-pattern factor information RPF_INF2 may include a second reference pattern factor value PF_REF2. In this case, the first reference pattern factor value PF_REF1 may be different from the second reference pattern factor value PF_REF2, and, for example, the first reference pattern factor value PF_REF1 may be greater than the second reference pattern factor value PF_REF2.

Referring to FIG. 8B, the simulation device 100 may generate combined reaction count-pattern factor information RPF_INF_CB, based on the first reaction count-pattern factor information RPF_INF1 and the second reaction count-pattern factor information RPF_INF2.

After generating the combined reaction count-pattern factor information RPF_INF_CB, the simulation device 100 may generate the pattern factor database 132 corresponding to the combined reaction count-pattern factor information RPF_INF_CB.

The combined reaction count-pattern factor information RPF_INF_CB may represent the reaction count-pattern factor information corresponding to a mathematical combination of the first reaction count-pattern factor information RPF_INF1 and the second reaction count-pattern factor information RPF_INF2. The combined reaction count-pattern factor information RPF_INF_CB may represent inherent information of the wafer WF that simulates a specific real wafer.

A reference pattern factor value corresponding to the combined reaction count-pattern factor information RPF_INF_CB may include a combined reference pattern factor value PF_REF_CB. The combined reference pattern factor value PF_REF_CB may be different from the first reference pattern factor value PF_REF1 and the second reference pattern factor value PF_REF2. For example, the combined reference pattern factor value PF_REF_CB may be less than the first reference pattern factor value PF_REF1 and greater than the second reference pattern factor value PF_REF2.

In an implementation, the “mathematical combination” of the first reaction count-pattern factor information RPF_INF1 and the second reaction count-pattern factor information RPF_INF2 may represent an operation used to couple the first reaction count-pattern factor information RPF_INF1 to the second reaction count-pattern factor information RPF_INF2. This operation may include, for example, weighted sums, simple addition or subtraction, averages (an arithmetic mean, a geometric mean, etc.), minimum values, maximum values, multiplication or exponentiation, logarithmic transformation, or a combination thereof. That is, coefficients or functions are applied to data points included in the first reaction count-pattern factor information RPF_INF1 and the second reaction count-pattern factor information RPF_INF2, and a set of calculation results generated by combining the data points to which the coefficients or functions have been applied may be referred to as the “mathematical combination.” However, the implementation is not limited to the examples described above and may include all mathematical and algorithmic processing methods recognized as common techniques in the art.

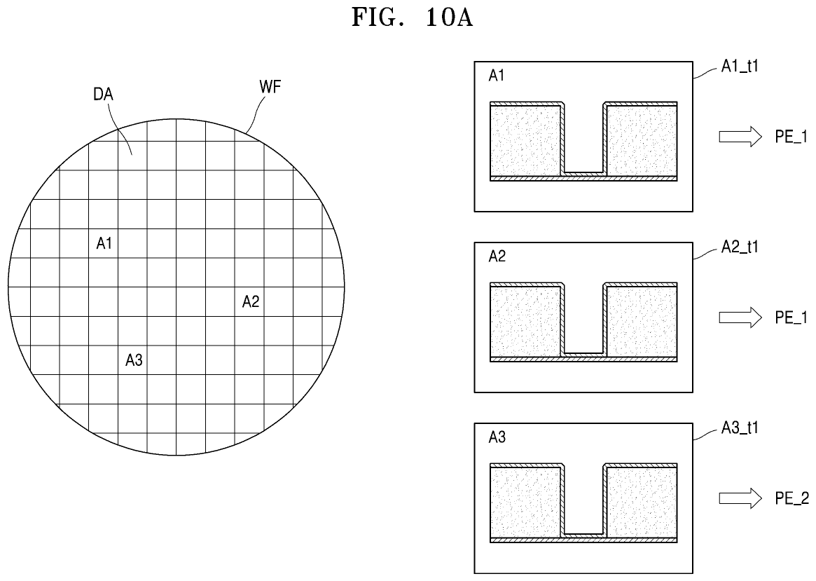

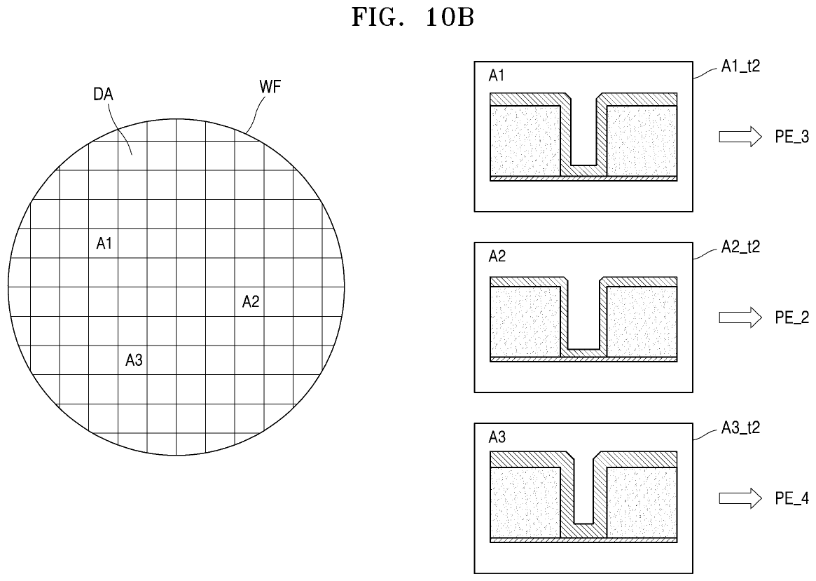

FIG. 9 is a flowchart illustrating a semiconductor process simulation method according to some implementations. Specifically, FIG. 9 may be a flowchart illustrating operation S230 of FIG. 5. FIGS. 10A and 10B are diagrams illustrating the reactor scale simulation performed based on the pattern factor database 132 and the reactor scale modeling parameters RSP. Specifically, FIG. 10A may show the wafer WF at the first point in time in the reactor scale simulation. The first point in time may represent the initial state of the reactor scale simulation (i.e., the state in which no film has been formed on the surface of the wafer WF). FIG. 10B may show the wafer WF at the second point in time in the reactor scale simulation. In this case, the second point in time may represent a point in time after the first point in time. FIGS. 9 to 10B may be described with reference to FIGS. 1 to 3, 4A to 4D, 5, 6, and 7A to 7E, and repeated descriptions thereof may be omitted.

Referring to FIGS. 10A and 10B, the wafer WF may be divided into a plurality of divided regions DA in operation S220 in FIG. 5. The plurality of divided regions DA may include a first region A1, a second region A2, and a third region A3. The first region A1, the second region A2, and the third region A3 may represent three arbitrary regions selected from among the plurality of divided regions DA, and hereinafter, these three regions are mainly described for convenience of description.

Referring to FIGS. 9 and 10A, in operation S231, the simulation device 100 may detect a plurality of pattern factor values corresponding to the reaction counts of each of the plurality of divided regions DA from the pattern factor database 132.

In an implementation, since the wafer WF at the first point in time is in the initial state in which no process has been performed, the surfaces of the first region A1, the second region A2, and the third region A3 may be as shown in FIG. 10A. That is, the first region A1 may be in a first region state A1_t1, the second region A2 may be in a second region state A2_t1, and the third region A3 may be in a third region state A3_t1.

In an implementation, based on the reaction count value in the first region A1 at the first point in time, the simulation device 100 may detect the first pattern factor value PF_1 from the pattern factor database 132 as the pattern factor value corresponding to the first region A1 at the first point in time.

In an implementation, based on the reaction count value in the second region A2 at the first point in time, the simulation device 100 may detect the first pattern factor value PF_1 from the pattern factor database 132 as the pattern factor value corresponding to the second region A2 at the first point in time.

In an implementation, based on the reaction count value in the third region A3 at the first point in time, the simulation device 100 may detect the first pattern factor value PF_1 from the pattern factor database 132 as the pattern factor value corresponding to the third region A3 at the first point in time.

Referring to FIG. 9, in operation S232, the simulation device 100 may update the boundary conditions of the reactor scale simulation based on the results detected in operation S231.

In an implementation, the boundary conditions of the reactor scale simulation may include pattern factor values corresponding to each of the plurality of divided regions DA. For example, the boundary conditions may include a pattern factor value corresponding to the first region A1, a pattern factor value corresponding to the second region A2, and a pattern factor value corresponding to the third region A3.

Referring to FIGS. 9 and 10B, in operation S233, the simulation device 100 may perform the reactor scale simulation based on the boundary conditions updated in operation S232, and may calculate the reaction count for each of the plurality of divided regions DA.

In an implementation, for each of the plurality of divided regions DA, the simulation device 100 may calculate the reaction count by performing the operation according to [Equation 1] based on the boundary conditions updated in operation S232. For example, in operation S232, the pattern factor value corresponding to the first region A1 may have the first pattern factor value PF_1. The simulation device 100 may input the first pattern factor value PF_1 to [Equation 1] as the pattern factor value and perform the operation described above. The same operation may be performed on each of the second region A2 and the third region A3, and repeated descriptions thereof may be omitted.

In an implementation, since the process has been performed on the wafer WF at the second point in time, the surfaces of the first region A1, the second region A2, and the third region A3 may be as shown in FIG. 10B. That is, at the second point in time, the first region A1 may be in a first region state A1_t2, the second region A2 may be in a second region state A2_t2, and the third region A3 may be in a third region state A3_t2.

In an implementation, since various variables are considered in the reactor scale simulation process, results according to [Equation 1] may vary depending on the position of the wafer WF even at the same point in time. For example, as shown in FIG. 10B, the thicknesses of films formed in the first region A1, the second region A2, and the third region A3 measured at the second point in time may be different from each other. For example, the thickness of the film formed in the first region A1 may be greater than the thickness of the film formed in the second region A2, and may be less than the thickness of the film formed in the third region A3. In other words, the cumulative reaction count generated in the first region A1 until the second point in time may be greater than the cumulative reaction count generated in the second region A2, and may be less than the cumulative reaction count generated in the third region A3.

In operation S234, the simulation device 100 may repeat the methods according to operations S231 to S233. In this case, the number of iterations may be determined according to the preset reference value of iterations.

In an implementation, the simulation device 100 may calculate the reaction count at the third point in time based on the reaction count calculated in operation S233. For example, the simulation device 100 may perform operation S231 again by detecting, from the pattern factor database 132, the third pattern factor value PF_3 as the pattern factor value corresponding to the first region A1, detecting the second pattern factor value PF_2 as the pattern factor value corresponding to the second region A2, and detecting the fourth pattern factor value PF 4 as the pattern factor value corresponding to the third region A3. Then, the simulation device 100 may perform processes according to operations S232 and S233 again.

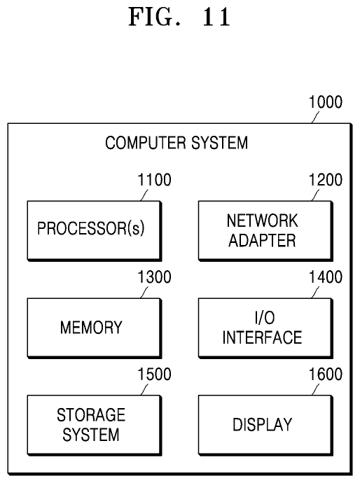

FIG. 11 is a block diagram illustrating a computer system 1000 according to an implementation.

The computer system 1000 in FIG. 11 may correspond to a layout simulation system described above with reference to the diagrams.

The computer system 1000 may represent any system that includes a universal or specialized computing system. For example, the computer system 1000 may include a personal computer, a server computer, a laptop computer, a home appliance, and the like. As shown in FIG. 11, the computer system 1000 may include at least one processor 1100, a network adapter 1200, memory 1300, an I/O interface 1400, a storage system 1500, and a display 1600.

The at least one processor 1100 may execute a program module that includes computer system-executable commands. The program module may include routines, programs, objects, components, logic, data structures, etc., which perform specific tasks or implement specific types of abstract data. The memory 1300 may include a computer system-readable medium in the form of volatile memory, such as random-access memory (RAM). The at least one processor 1100 may access the memory 1300 and execute commands loaded in the memory 1300. The storage system 1500 may store information in a non-volatile manner and include at least one program product that includes a program module configured to perform training of machine learning models for the layout simulation described above with reference to the diagrams in some implementations. The program may include, as a non-limiting example, an operating system, at least one application, other program modules, and program data.

The network adapter 1200 may provide access to a local area network (LAN), a wide area network (WAN), and/or a public network (e.g., the Internet). The I/O interface 1400 may provide channels for communicating with peripheral devices, such as a keyboard, a pointing device, and an audio system. The display 1600 may output various pieces of information for a user to see.

In some implementations, the semiconductor process simulation method described above with reference to the diagrams may be implemented as a computer program product. The computer program product may include a non-transitory computer-readable medium (or a storage medium) that includes computer-readable program commands for making at least one processor 1100 perform image processing and/or training of models. The computer-readable commands may include, as a non-limiting example, an assembler command, an instruction set architecture (ISA) command, a machine command, a machine-dependent command, microcode, a firmware command, status setting data, or source code or object code written in at least one programming language.

The computer-readable medium may include any medium capable of holding and storing, in a non-transitory manner, commands that are executed by at least one processor 1100 or any command execution device. The computer-readable medium may include, but is not limited to, an electronic storage device, a magnetic storage device, an optical storage device, an electromagnetic storage device, a semiconductor storage device, or any combination thereof. For example, the computer-readable media may include a portable computer diskette, a hard disc, RAM, read-only memory (ROM), electrically erasable read-only memory (EEPROM), flash memory, static random-access memory (SRAM), a compact disc (CD), a digital versatile disc (DVD), a memory stick, a floppy disc, a mechanically encoded device such as a punch card, or any combination thereof.

While the present disclosure contains many specific implementation details, these should not be construed as limitations on the scope of any invention or on the scope of what may be claimed, but rather as descriptions of features that may be specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a combination can in some cases be excised from the combination, and the combination may be directed to a subcombination or variation of a subcombination.

While the present disclosure has been particularly shown and described with reference to implementations thereof, it will be understood that various changes in form and details may be made therein without departing from the spirit and scope of the following claims.

What is claimed is:

1. A method comprising:
generating a pattern factor database based on reaction count-pattern factor information that represents an available reaction amount of a pattern according to a reaction count of a chemical reaction occurring on a surface of a target pattern;
dividing a wafer region into a plurality of divided regions; and
generating a reactor scale simulation result by repeatedly performing a reactor scale simulation based on the pattern factor database,
wherein the reactor scale simulation comprises:
detecting, from the pattern factor database, a plurality of pattern factor values corresponding to reaction counts of each of the plurality of divided regions;
updating a boundary condition of the reactor scale simulation based on the detected pattern factor values; and
performing the reactor scale simulation based on the updated boundary condition and calculating the reaction counts for each of the plurality of divided regions.

2. The method of claim 1, wherein the pattern factor database comprises a plurality of pattern factor entries, and
each of the plurality of pattern factor entries comprises:
a reaction count value representing a number of cumulative reaction particles of the chemical reaction generated on the surface of the target pattern upto a specific point in time; and
a pattern factor value representing an available reaction amount of a pattern corresponding to the reaction count value.

3. The method of claim 1, wherein generating the pattern factor database comprises:
generating first reaction count-pattern factor information corresponding to a first target pattern;
generating second reaction count-pattern factor information corresponding to a second target pattern having a different pattern from the first target pattern; and
generating combined reaction count-pattern factor information corresponding to a mathematical combination of the first reaction count-pattern factor information and the second reaction count-pattern factor information, and
wherein a reference pattern factor value of the first reaction count-pattern factor information is different from a reference pattern factor value of the second reaction count-pattern factor information.

4. The method of claim 1, wherein generating the pattern factor database comprises:
generating first reaction count information by simulating the chemical reaction based on a flat pattern, wherein the first reaction count information represents a reaction count occurring on a surface of the flat pattern according to a simulation elapsed time;
generating second reaction count information by simulating the chemical reaction based on the target pattern, wherein the second reaction count information represents a reaction count occurring on the surface of the target pattern according to the simulation elapsed time;
generating first reaction rate information corresponding to the first reaction count information and generating second reaction rate information corresponding to the second reaction count information;
generating time-pattern factor information, based on the first reaction rate information and the second reaction rate information, wherein the time-pattern factor information represents pattern factor values according to the simulation elapsed time;
generating the reaction count-pattern factor information by replacing the simulation elapsed time with a cumulative reaction count in the time-pattern factor information, wherein the reaction count-pattern factor information represents a pattern factor value according to the cumulative reaction count; and
generating the pattern factor database based on the reaction count-pattern factor information.

5. The method of claim 1, wherein the reaction count of the chemical reaction is defined by one or more of:
a pressure value of a first material;
a pressure value of a second material;
activation energy of the chemical reaction; and
a pattern factor value.

6. The method of claim 1, wherein the boundary condition of the reactor scale simulation is defined by the plurality of pattern factor values corresponding to each of the plurality of divided regions.

7. The method of claim 1, wherein the reactor scale simulation result comprises reaction count information corresponding to each of the plurality of divided regions.

8. The method of claim 1, wherein the reactor scale simulation is performed repeatedly according to a preset reference value of iterations.

9. The method of claim 1, wherein the plurality of divided regions comprise a first divided region and a second divided region, and
wherein, for a first reactor scale simulation performed at a first time, a pattern factor value corresponding to the first divided region is equal to a pattern factor value corresponding to the second divided region.

10. The method of claim 9, wherein, for one or more reactor scale simulations performed after the first time, the pattern factor value corresponding to the first divided region is different from the pattern factor value corresponding to the second divided region.

11. A system comprising:
memory configured to store a program for performing a method; and
one or more processors configured to execute the program stored in the memory,
wherein the one or more processors are configured to:
generate a pattern factor database representing an available reaction amount of a pattern according to a reaction count of a chemical reaction occurring on a surface of a target pattern;
divide a wafer region into a plurality of divided regions; and
generate a reactor scale simulation result by repeatedly performing a reactor scale simulation based on the pattern factor database, and
wherein the reactor scale simulation comprises:
detect, from the pattern factor database, a plurality of pattern factor values corresponding to reaction counts of each of the plurality of divided regions;
update a boundary condition of the reactor scale simulation based on the detected pattern factor values; and
perform the reactor scale simulation based on the updated boundary condition and calculate the reaction counts for each of the plurality of divided regions.

12. The system of claim 11, wherein the pattern factor database comprises a plurality of pattern factor entries, and
wherein each of the plurality of pattern factor entries comprises:
a reaction count value representing a number of cumulative reaction particles of the chemical reaction generated on the surface of the target pattern until a specific point in time; and
a pattern factor value representing an available reaction amount of a pattern corresponding to the reaction count value.

13. The system of claim 12, wherein the one or more processors are configured to:
generate first reaction count information by simulating the chemical reaction based on a flat pattern, wherein the first reaction count information represents a reaction count occurring on a surface of the flat pattern according to a simulation elapsed time;
generate second reaction count information by simulating the chemical reaction based on the target pattern, wherein the second reaction count information represents a reaction count occurring on the surface of the target pattern according to the simulation elapsed time;
generate first reaction rate information corresponding to the first reaction count information and generate second reaction rate information corresponding to the second reaction count information;
generate time-pattern factor information, based on the first reaction rate information and the second reaction rate information, wherein the time-pattern factor information represents pattern factor values according to the simulation elapsed time;
generate reaction count-pattern factor information by replacing the simulation elapsed time with a cumulative reaction count in the time-pattern factor information, wherein the reaction count-pattern factor information represents a pattern factor value according to the cumulative reaction count; and
generate the pattern factor database based on the reaction count-pattern factor information.

14. The system of claim 11, wherein the plurality of divided regions comprise a first divided region and a second divided region, and
wherein, for a first reactor scale simulation performed at a first time, a pattern factor value corresponding to the first divided region is equal to a pattern factor value corresponding to the second divided region.

15. The system of claim 14, wherein, for one or more reactor scale simulations performed after the first time, the pattern factor value corresponding to the first divided region is different from the pattern factor value corresponding to the second divided region.

16. A computer-readable non-transitory storage medium configured to store commands, when executed by one or more processors, to make the one or more processors perform a method,
wherein the method comprises:
generating a pattern factor database representing an available reaction amount of a pattern according to a reaction count of a chemical reaction occurring on a surface of a target pattern;
dividing a wafer region into a plurality of divided regions; and
generating a simulation result by repeatedly performing a simulation based on the pattern factor database,
wherein generating the simulation result comprises:
detecting, from the pattern factor database, a plurality of pattern factor values corresponding to reaction counts of each of the plurality of divided regions;
updating a boundary condition of the simulation based on the detected pattern factor values; and
performing the simulation based on the updated boundary condition and calculating the reaction counts for each of the plurality of divided regions.

17. The computer-readable non-transitory storage medium of claim 16, wherein the pattern factor database comprises a plurality of pattern factor entries, and
wherein each of the plurality of pattern factor entries comprises:
a reaction count value representing a number of cumulative reaction particles of the chemical reaction generated on the surface of the target pattern until a specific point in time; and
a pattern factor value representing an available reaction amount of a pattern corresponding to the reaction count value.

18. The computer-readable non-transitory storage medium of claim 16, wherein generating the pattern factor database comprises:
generating first reaction count information by simulating the chemical reaction based on a flat pattern, wherein the first reaction count information represents a reaction count occurring on a surface of the flat pattern according to a simulation elapsed time;
generating second reaction count information by simulating the chemical reaction based on the target pattern, wherein the second reaction count information represents a reaction count occurring on the surface of the target pattern according to the simulation elapsed time;
generating first reaction rate information corresponding to the first reaction count information and generating second reaction rate information corresponding to the second reaction count information;
generating time-pattern factor information, based on the first reaction rate information and the second reaction rate information, wherein the time-pattern factor information represents pattern factor values according to the simulation elapsed time;
generating reaction count-pattern factor information by replacing the simulation elapsed time with a cumulative reaction count in the time-pattern factor information, wherein the reaction count-pattern factor information represents a pattern factor value according to the cumulative reaction count; and
generating the pattern factor database based on the reaction count-pattern factor information.

19. The computer-readable non-transitory storage medium of claim 16, wherein the plurality of divided regions comprise a first divided region and a second divided region, and
wherein for a first simulation performed at a first time,
a pattern factor value corresponding to the first divided region is equal to a pattern factor value corresponding to the second divided region.

20. The computer-readable non-transitory storage medium of claim 19, wherein, for one or more simulations performed after the first time, the pattern factor value corresponding to the first divided region is different from the pattern factor value corresponding to the second divided region.
